SEQUENCE LISTING (3 sequences)

SEQ ID NO: 1 - DNA, 32 bp, Artificial
  hypothetical sequence for an illustration
actatgacgctttccatccgggctagctctca

SEQ ID NO: 2 - RNA, 21 nt, Artificial
  hypothetical RNA for illustration
acuaugacgcuuuccaucggg

SEQ ID NO: 3 - protein, 6 aa, Artificial
  hypothetical protein sequence for illustration
YDAFHR

(19) United States
(12) Patent Application Publication
(10) Pub. No.: US 2006/0084067 A1
(43) Pub. Date: Apr. 20, 2006

(54) Method and system for analysis of array-based, comparative-hybridization data

(76) Inventors: Zohar Yakhini, Ramat Hasharon (IL); Amir Ben-Dor, Bellevue, WA (US); Robert Kincaid, Half Moon Bay, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/953,958

(22) Filed: Sep. 29, 2004

Related U.S. Application Data
(60) Provisional application No. 60/541,711, filed on Feb. 3, 2004.

(51) Int. Cl. C12Q 1/68; G06F 19/00

(52) U.S. Cl. 435/6; 702/20

(57) ABSTRACT

Embodiments of the present invention include methods and systems for analysis of comparative genomic hybridization (“CGH”) data, including CGH data obtained from microarray experiments. Various embodiments of the present invention include parametric and non-parametric normalization methods for CGH data, methods for identifying sets of one or more contiguous chromosomal DNA subsequences that are amplified or deleted in cells from particular tissue samples, and methods for determining amplifications and deletions common to a set of analyzed samples. When combined with well-designed microarray-based experimental systems, method embodiments of the present invention provide markedly increased quantitative precision in the identification of chromosomal abnormalities, including amplified and deleted DNA subsequences based on CGH data.

### CROSS-REFERENCE TO RELATED APPLICATION

This application claims the benefit of provisional application No. 60/541,711, filed Feb. 3, 2004

The present invention is related to analysis of experimental data and, in particular, to a method and system for identifying biopolymer-sequence abnormalities, including amplifications and deletions of subsequences of the DNA sequence of a chromosomal DNA, in samples of interest compared to control samples by array-based comparative hybridization.

### BACKGROUND OF THE INVENTION

A great deal of basic research has been carried out to elucidate the causes and cellular mechanisms responsible for transformation of normal cells to a precancerous or cancerous state, and for the growth of cancerous tissues and metastasis of cancerous tissues. Enormous strides have been made in understanding various causes and cellular mechanisms of cancer, and this detailed understanding is currently providing new and useful approaches for preventing, detecting, and treating cancer.

There are myriad different types of causative events and agents associated with the development of cancer. Moreover, there are many different types of cancer, and many different patterns of cancer development for each of the many different types of cancer. Although initial hopes and strategies were predicated on finding one or a few basic, underlying causes and mechanisms, researchers have, over time, recognized that, in fact, the term “cancer” encompasses a very large number of different diseases. Nonetheless, there do appear to be certain common cellular phenomena associated with cancer. One common phenomenon, evident in many different types of cancer, is the onset of genetic instability in precancerous tissues, and progressive genomic instability as cancerous tissue develops. While there are many different types and manifestations of genomic instability, a change in the number of copies of particular DNA subsequences within a cancerous cell may be a fundamental indication of genomic instability. Various techniques have been developed to detect and at least partially quantify amplification and deletion of chromosomal DNA subsequences in cancerous cells. One technique is referred to as “comparative genomic hybridization.” Comparative genomic hybridization (“CGH”) can offer striking, visual indications of chromosomal-DNA-subsequence amplification and deletion, in certain cases, but, like many biological and biochemical analysis techniques, is subject to significant noise and sample variation, leading to problems in quantitative analysis of CGH data. Research scientists, diagnosticians, and medical personnel have recognized the need for CGH-data analysis techniques to more accurately quantify DNA-subsequence-copy variation in diseased tissue samples, including cancerous cells, as well as techniques for analyzing CGH-data, and visualizing analytical results, obtained by applying CGH techniques to samples from multiple sources in order to identify possible genetic bases for various observed characteristics and conditions related to the sources.

### SUMMARY OF THE INVENTION

Embodiments of the present invention include methods and systems for analysis of comparative hybridization data, including comparative genomic hybridization (“CGH”) data, such as CGH data obtained from microarray experiments. Various embodiments of the present invention include parametric and non-parametric normalization methods for CGH data and methods for identifying sets of one or more contiguous chromosomal DNA subsequences that are amplified or deleted in cells from particular tissue samples. When combined with well-designed microarray-based experimental systems, method embodiments of the present invention provide markedly increased quantitative precision in the identification of chromosomal abnormalities, including amplified and deleted DNA subsequences based on CGH data. Additional embodiments of the present invention are directed to detecting, by comparative hybridization, deletion, amplifications, and other changes to general biopolymer sequences, including biopolymers other than DNA.

### BRIEF DESCRIPTION OF THE DRAWINGS

FIG. 1 shows the chemical structure of a small, four-subunit, single-chain oligonucleotide.

FIG. 2 shows a symbolic representation of a short stretch of double-stranded DNA.

FIG. 3 illustrates construction of a protein based on the information encoded in a gene.

FIG. 4 shows a hypothetical set of chromosomes for a very simple, hypothetical organism.

FIG. 5 shows examples of gene deletion and gene amplification in the context of the hypothetical genome shown in FIG. 4.

FIGS. 6-7 illustrate detection of gene amplification by CGH.

FIGS. 8-9 illustrate detection of gene deletion by CGH.

FIGS. 10-11 illustrate microarray-based CGH.

FIGS. 12-16 show data that illustrates the number of combinations of gene-rank values that lead to a particular rank (I) value for a number of genes in an interval and an arbitrary number of samples.

FIG. 17 illustrates one method for identifying and ranking intervals and removing redundancies from lists of intervals identified as probable deletions or amplifications.

FIGS. 18A-F show screen captures that illustrate a user interface developed to provide visual and interactive access to methods of CGH data analysis and results of the analysis as part of a CGH-data-analysis system.

### DETAILED DESCRIPTION OF THE INVENTION

Embodiments of the present invention provide methods and systems for analysis of comparative genomic hybridization (“CGH”) data. The methods and systems are general, and applicable to comparative hybridization data obtained from a variety of different experimental approaches and protocols. Described embodiments, below, are particularly applicable to microarray-based CGH data, obtained from high-resolution microarrays containing oligonucleotide probes that provide relatively uniform and closely-spaced coverage of the DNA sequence or sequences representing one or more chromosomes. One application for methods of the present invention is for detecting amplified and deleted genes. Examples are discussed below. However, any subsequence of chromosomal DNA may be amplified or deleted, and CGH techniques may be applied to generally detect amplification or deletion of chromosomal DNA subsequences. Comparative hybridization methods can be used to detect amplification or deletion of subsequences of any information-containing biopolymer, and other sequence changes and abnormalities.

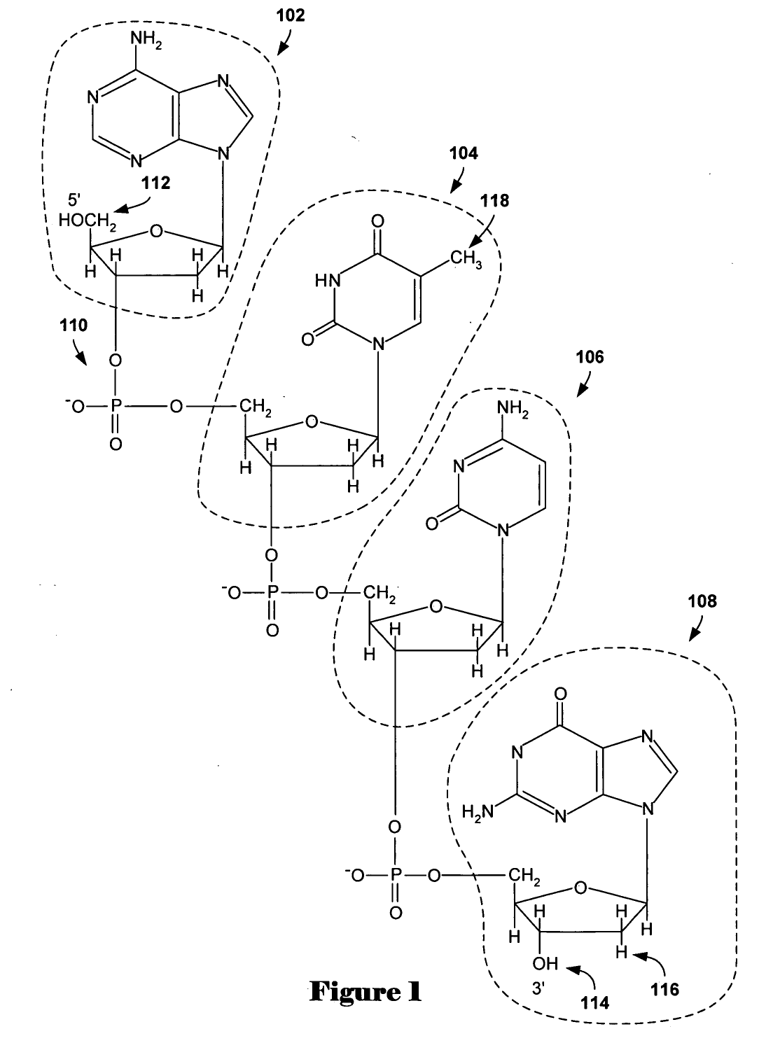

Prominent information-containing biopolymers include deoxyribonucleic acid (“DNA”), ribonucleic acid (“RNA”), including messenger RNA (“mRNA”), and proteins. FIG. 1 shows the chemical structure of a small, four-subunit, single-chain oligonucleotide, or short DNA polymer. The oligonucleotide shown in FIG. 1 includes four subunits: (1) deoxyadenosine 102, abbreviated “A”; (2) deoxythymidine 104, abbreviated “T”; (3) deoxycytodine 106, abbreviated “C”; and (4) deoxyguanosine 108, abbreviated “G.” Each subunit 102, 104, 106, and 108 is generically referred to as a “deoxyribonucleotide,” and consists of a purine, in the case of A and G, or pyrimidine, in the case of C and T, covalently linked to a deoxyribose. The deoxyribonucleotide subunits are linked together by phosphate bridges, such as phosphate 110. The oligonucleotide shown in FIG. 1, and all DNA polymers, is asymmetric, having a 5′ end 112 and a 3′ end 114, each end comprising a chemically active hydroxyl group. RNA is similar, in structure, to DNA, with the exception that the ribose components of the ribonucleotides in RNA have a 2′ hydroxyl instead of a 2′ hydrogen atom, such as 2′ hydrogen atom 116 in FIG. 1, and include the ribonucleotide uridine, similar to thymidine but lacking the methyl group 118, instead of a ribonucleotide analog to deoxythymidine. The RNA subunits are abbreviated A, U, C, and G.

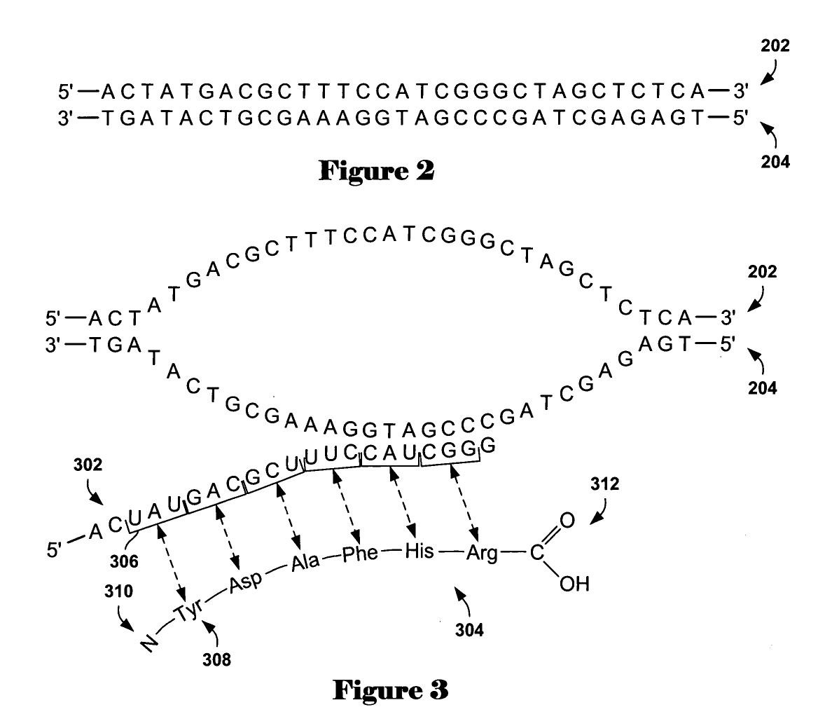

In cells, DNA is generally present in double-stranded form, in the familiar DNA-double-helix form. FIG. 2 shows a symbolic representation of a short stretch of double-stranded DNA. The first strand 202 is written as a sequence of deoxyribonucleotide abbreviations in the 5′ to 3′ direction and the complementary strand 204 is symbolically written in 3′ to 5′ direction. Each deoxyribonucleotide subunit in the first strand 202 is paired with a complementary deoxyribonucleotide subunit in the second strand 204. In general, a G in one strand is paired with a C in a complementary strand, and an A in one strand is paired with a T in a complementary strand. One strand can be thought of as a positive image, and the opposite, complementary strand can be thought of as a negative image, of the same information encoded in the sequence of deoxyribonucleotide subunits.

A gene is a subsequence of deoxyribonucleotide subunits within one strand of a double-stranded DNA polymer. A gene can be thought of as an encoding that specifies, or a template for, construction of a particular protein. FIG. 3 illustrates construction of a protein based on the information encoded in a gene. In a cell, a gene is first transcribed into single-stranded MRNA. In FIG. 3, the double-stranded DNA polymer composed of strands 202 and 204 has been locally unwound to provide access to strand 204 for transcription machinery that synthesizes a single-stranded mRNA 302 complementary to the gene-containing DNA strand. The single-stranded MRNA is subsequently translated by the cell into a protein polymer 304, with each three-ribonucleotide codon, such as codon 306, of the mRNA specifying a particular amino acid subunit of the protein polymer 304. For example, in FIG. 3, the codon “UAU” 306 specifies a tyrosine amino-acid subunit 308. Like DNA and RNA, a protein is also asymmetrical, having an N-terminal end 310 and a carboxylic acid end 312.

In eukaryotic organisms, including humans, each cell contains a number of extremely long, DNA-double-strand polymers called chromosomes. Each chromosome can be thought of, abstractly, as a very long deoxyribonucleotide sequence. Each chromosome contains hundreds to thousands of subsequences corresponding to genes. The exact correspondence between a particular subsequence identified as a gene and the protein encoded by the gene can be somewhat complicated, for reasons outside the scope of the present invention. However, for the purposes of describing embodiments of the present invention, a chromosome may be thought of as a linear DNA sequence of contiguous deoxyribonucleotide subunits that can be viewed as a linear sequence of DNA subsequences. In certain cases, the subsequences are genes, each gene specifying a particular protein. But these embodiments are far more general. Amplification and deletion of any DNA subsequence or group of DNA subsequences can be detected by the described methods, regardless of whether or not the DNA subsequences correspond to protein-sequence-specifying, biological genes, to DNA subsequences specifying various types of non-protein-encoding RNAs, or to other regions with defined biological roles. Moreover, these methods may be applied to other types of biopolymers to detect changes in biopolymer-subsequence occurrence. The term “gene” is used in the following as a notational convenience, and should be understood as simply an example of a “biopolymer subsequence.” Similarly, although the described embodiments are directed to analyzing DNA chromosomal sequences, the sequences of any information-containing biopolymer are analyzable by methods of the present invention. Therefore, the term “chromosome,” and related terms, are used in the following as a notational convenience, and should be understood as an example of a biopolymer or biopolymer sequence.

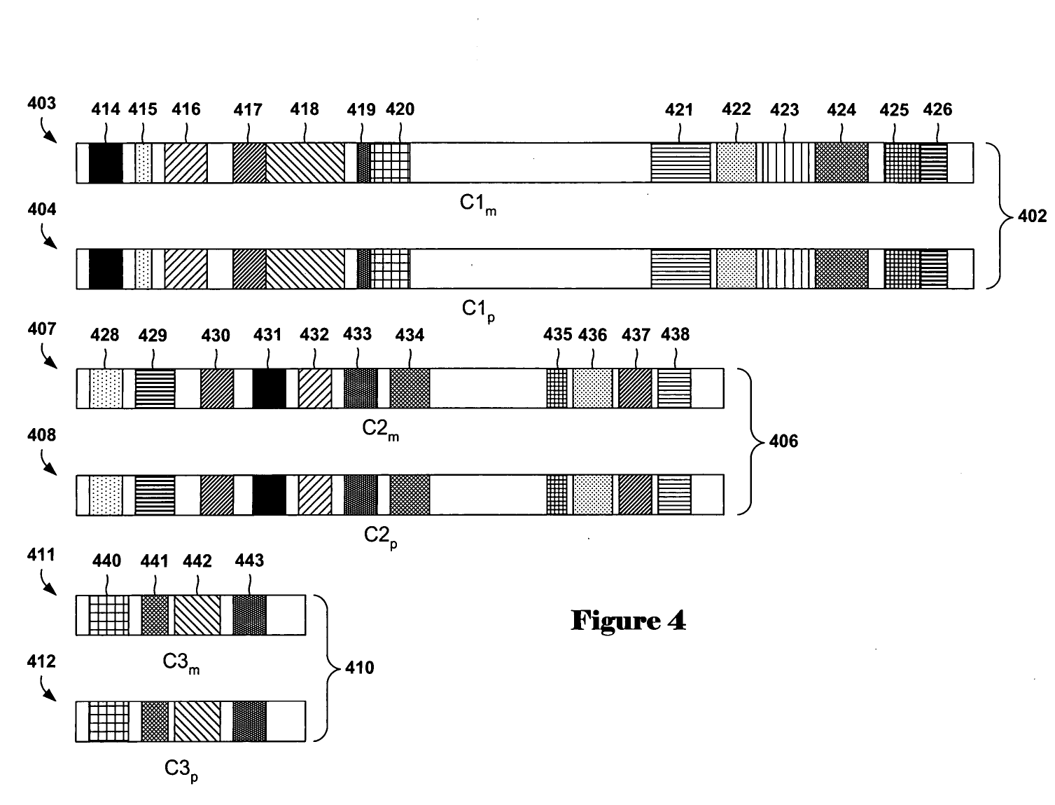

FIG. 4 shows a hypothetical set of chromosomes for a very simple, hypothetical organism. The hypothetical organism includes three pairs of chromosomes 402, 406, and 410. Each chromosome in a pair of chromosomes is quite similar, generally having identical genes at identical positions along the lines of the chromosome. In FIG. 4, each gene is represented as a subsection of the chromosome. For example, in the first chromosome 403 of the first chromosome pair 402, 13 genes are shown, 414-426.

As shown in FIG. 4, the second chromosome 404 of the first pair of chromosomes 402 includes the same genes at the same positions. Each chromosome of the second pair of chromosomes 406 includes eleven genes 428-438, and each chromosome of the third pair of chromosomes 410 includes four genes 440-443. Of course, in a real organism, there are generally many more chromosome pairs, and each chromosome includes many more genes. However, the simplified, hypothetical genome shown in FIG. 4 is more suitable for simply describing embodiments of the present invention. Note that, in each chromosome pair, one chromosome is originally obtained from the mother of the organism, and the other chromosome is originally obtained from the father of the organism. Thus, the chromosomes of the first chromosome pair 402 are referred to as chromosome “C1ₘ” and “C1ₚ.” While, in general, each chromosome of a chromosome pair has the same genes positioned at the same location along the length of the chromosome, the genes inherited from one parent may differ slightly from the genes inherited from the other parent. Different versions of a gene are referred to as alleles. Common differences include single-deoxyribonucleotide-subunit substitutions at various positions within the DNA subsequence corresponding to a gene.

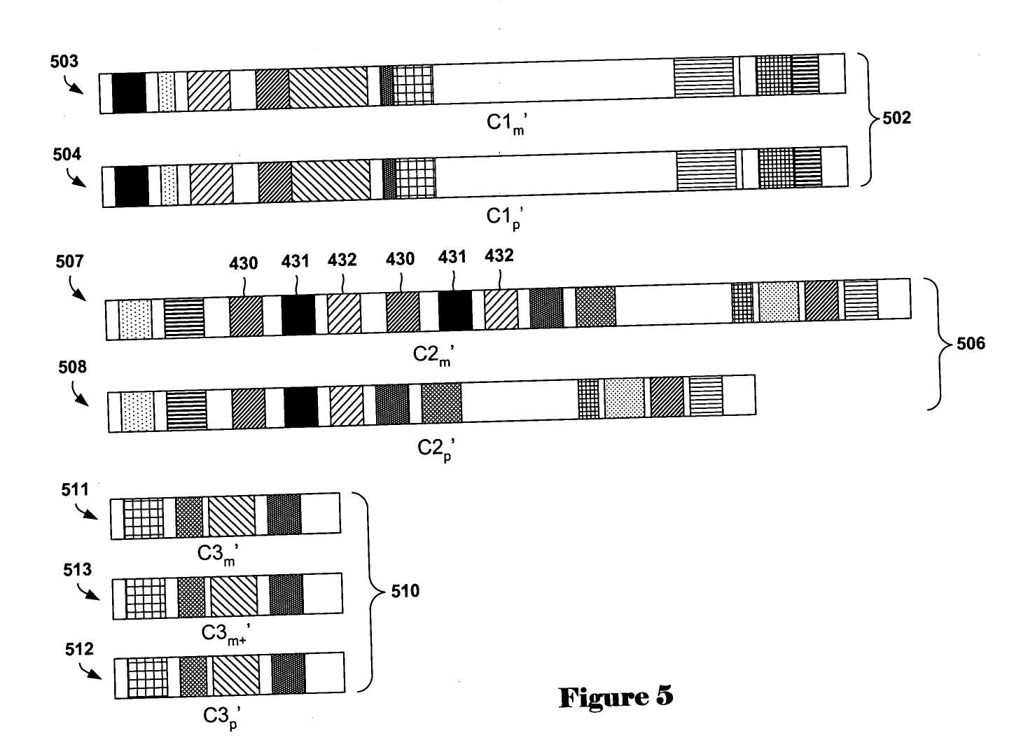

Although differences between genes and mutations of genes may be important in the predisposition of cells to various types of cancer, and related to cellular mechanisms responsible for cell transformation, cause-and-effect relationships between different forms of genes and pathological conditions are often difficult to elucidate and prove, and very often indirect. However, other genomic abnormalities are more easily associated with pre-cancerous and cancerous tissues. Two prominent types of genomic aberrations include gene amplification and gene deletion. FIG. 5 shows examples of gene deletion and gene amplification in the context of the hypothetical genome shown in FIG. 4. First, both chromosomes C1ₘ′ 503 and chromosome C1ₚ′ 504 of the variant, or mutant, first chromosome pair 502 are shorter than the corresponding wild-type chromosomes C1ₘ and C1ₚ in the first pair of chromosomes 402 shown in FIG. 4. This shortening is due to deletion of genes 422, 423, and 424, present in the wild-type chromosomes 403 and 404, but absent in the variant chromosomes 503 and 504. This is an example of a double, or homozygous-gene-deletion. Small scale variations of DNA copy numbers can also exist in normal cells. These can have phenotypic implications, and can also be measured by CGH methods and analyzed by the methods of the present invention.

Generally, deletion of multiple, contiguous genes is observed, corresponding to the deletion of a substantial subsequence from the DNA sequence of a chromosome. Much smaller subsequence deletions may also be observed, leading to mutant and often nonfunctional genes. A gene deletion may be observed in only one of the two chromosomes of a chromosome pair, in which case a gene deletion is referred to as being heterozygous. A second chromosomal abnormality in the altered genome shown in FIG. 5 is duplication of genes 430, 431, and 432 in the maternal chromosome C2ₘ′ 507 of the second chromosome pair 506. Duplication of one or more contiguous genes within a chromosome is referred to as gene amplification. In the example altered genome shown in FIG. 5, the gene amplification in chromosome C2ₘ′ is heterozygous, since gene amplification does not occur in the other chromosome of the pair C2ₚ′ 508. The gene amplification illustrated in FIG. 5 is a two-fold amplification, but three-fold and higher-fold amplifications are also observed. An extreme chromosomal abnormality is illustrated with respect to the third chromosome pair (410 in FIG. 4). In the altered genome illustrated in FIG. 5, the entire maternal chromosome 511 has been duplicated from a third chromosome 513, creating a chromosome triplet 510 rather than a chromosome pair. This three-chromosome phenomenon is referred to as a trisomy in the third chromosome-pair. The trisomy shown in FIG. 5 is an example of heterozygous gene amplification, but it is also observed that both chromosomes of a chromosome pair may be duplicated, higher-order amplification of chromosomes may be observed, and heterozygous and homozygous deletions of entire chromosomes may also occur, although organisms with such genetic deletions are generally not viable.

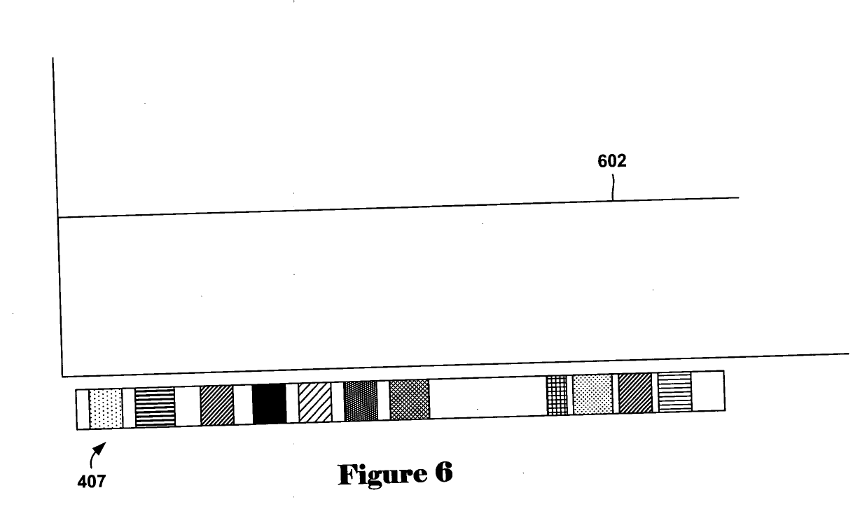

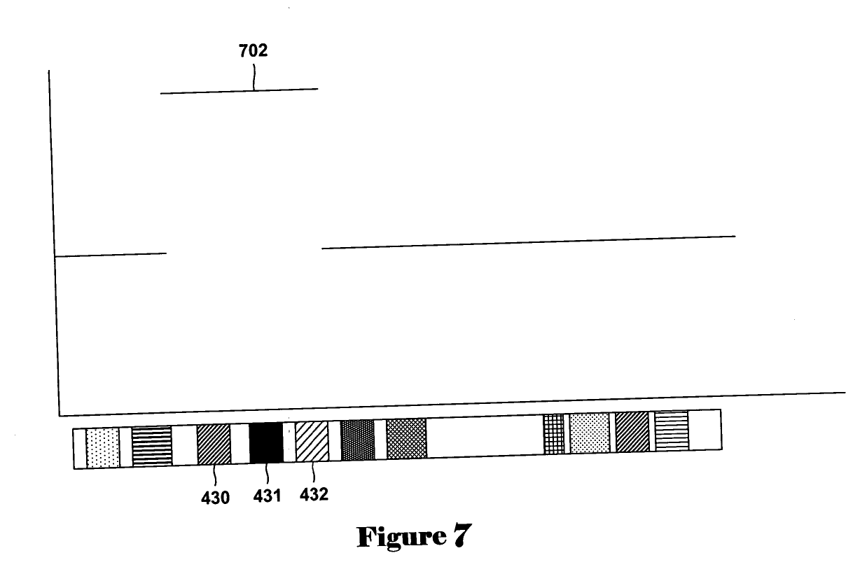

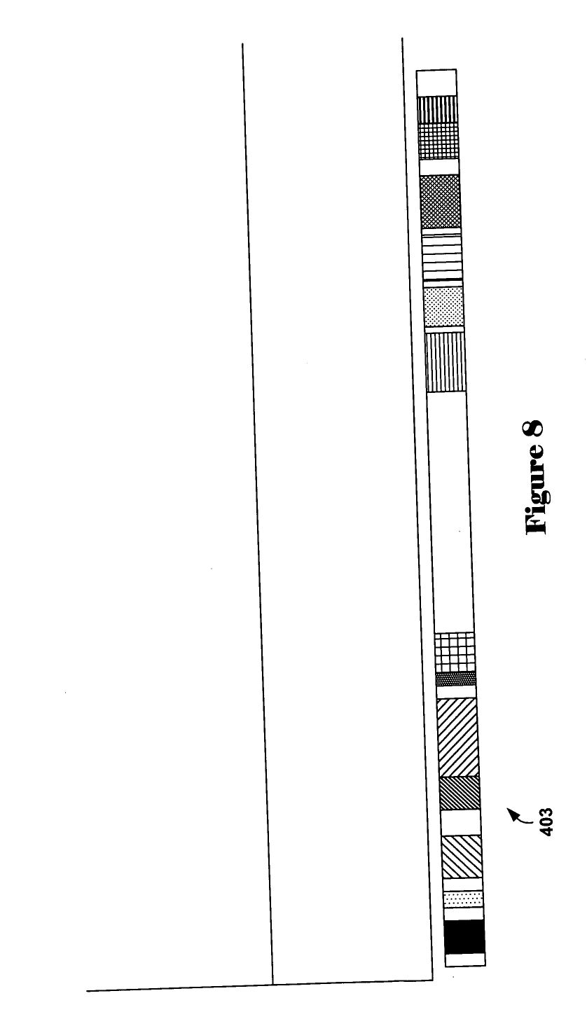

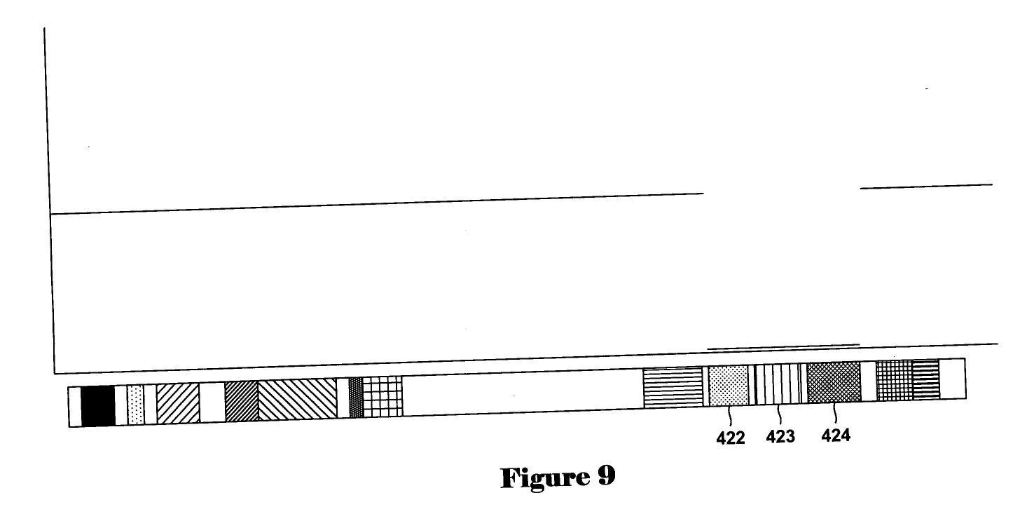

Changes in the number of gene copies, either by amplification or deletion, can be detected by comparative genomic hybridization (“CGH”) techniques. FIGS. 6-7 illustrate detection of gene amplification by CGH, and FIGS. 8-9 illustrate detection of gene deletion by CGH. CGH involves analysis of the relative level of binding of chromosome fragments from sample tissues to single-stranded, normal chromosomal DNA. The tissues-sample fragments hybridize to complementary regions of the normal, single-stranded DNA by complementary binding to produce short regions of double-stranded DNA. Hybridization occurs when a DNA fragment is exactly complementary, or nearly complementary, to a subsequence within the single-stranded chromosomal DNA. In FIG. 6, and in subsequent figures, one of the hypothetical chromosomes of the hypothetical wild-type genome shown in FIG. 4 is shown below the x axis of a graph, and the level of sample fragment binding to each portion of the chromosome is shown along with the y axis. In FIG. 6, the graph of fragment binding is a horizontal line 602 indicative of generally uniform fragment binding along the length of the chromosome 407. Of course, in an actual experiment, uniform and complete overlap of DNA fragments prepared from tissue samples may not be possible, leading to discontinuities and non-uniformities in detected levels of fragment binding along the length of a chromosome. However, in general, fragments of a normal chromosome isolated from normal tissue samples should, at least, provide a binding-level trend approaching a horizontal line, such as line 602 in FIG. 6. By contrast, CGH data for fragments prepared from the mutant genotype illustrated in FIG. 5 should generally show an increased binding level for those genes amplified in the mutant genotype.

FIG. 7 shows hypothetical CGH data for fragments prepared from tissues with the mutant genotype illustrated in FIG. 5. As shown in FIG. 7, an increased binding level 702 is observed for the three genes 430-432 that are amplified in the altered genome. In other words, the fragments prepared from the altered genome should be enriched in those gene fragments from genes which are amplified. Moreover, in quantitative CGH, the relative increase in binding should be reflective of the increase in a number of copies of particular genes.

FIG. 8 shows hypothetical CGH data for fragments prepared from normal tissue with respect to the first hypothetical chromosome 403. Again, the CGH-data trend expected for fragments prepared from normal tissue is a horizontal line indicating uniform fragment binding along the length of the chromosome. By contrast, the homozygous gene deletion in chromosomes 503 and 504 in the altered genome illustrated in FIG. 5 should be reflected in a relative decrease in binding with respect to the deleted genes. FIG. 9 illustrates hypothetical CGH data for DNA fragments prepared from the hypothetical altered genome illustrated in FIG. 5 with respect to a normal chromosome from the first pair of chromosomes (402 in FIG. 4). As seen in FIG. 9, no fragment binding is observed for the three deleted genes 422, 423, and 424.

CGH data may be obtained by a variety of different experimental techniques. In one technique, DNA fragments are prepared from tissue samples and labeled with a particular chromophore. The labeled DNA fragments are then hybridized with single-stranded chromosomal DNA from a normal cell, and the single-stranded chromosomal DNA then visually inspected via microscopy to determine the intensity of light emitted from labels associated with hybridized fragments along the length of the chromosome. Areas with relatively increased intensity reflect regions of the chromophore amplified in the corresponding tissue chromosome, and regions of decreased emitted signal indicate deleted regions in the corresponding tissue chromosome. In other techniques, normal DNA fragments labeled with a first chromophore are competitively hybridized to a normal single-stranded chromosome with fragments isolated from abnormal tissue, labeled with a second chromophore. Relative binding of normal and abnormal fragments can be detected by ratios of emitted light at the two different intensities corresponding to the two different chromophore labels.

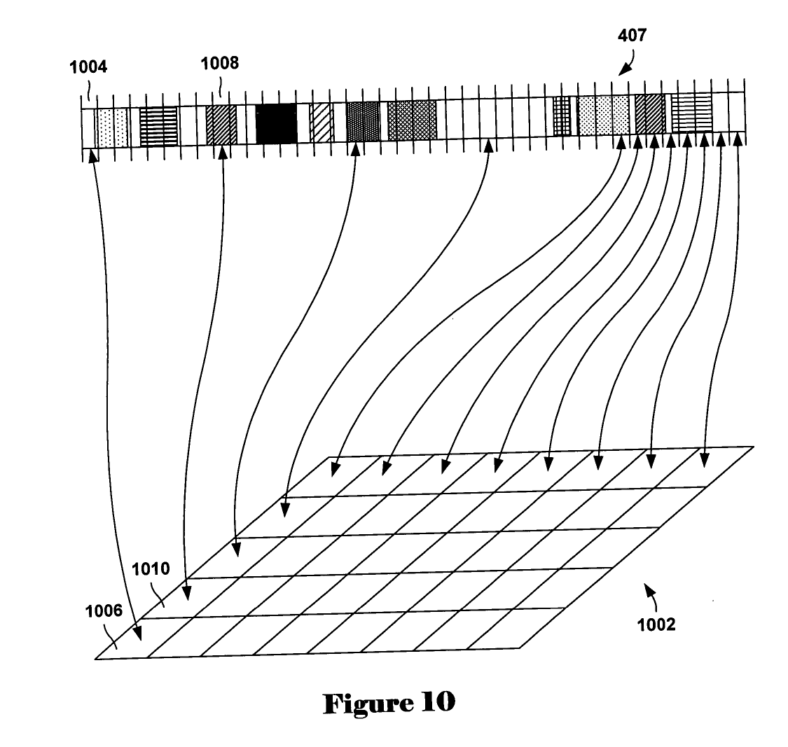

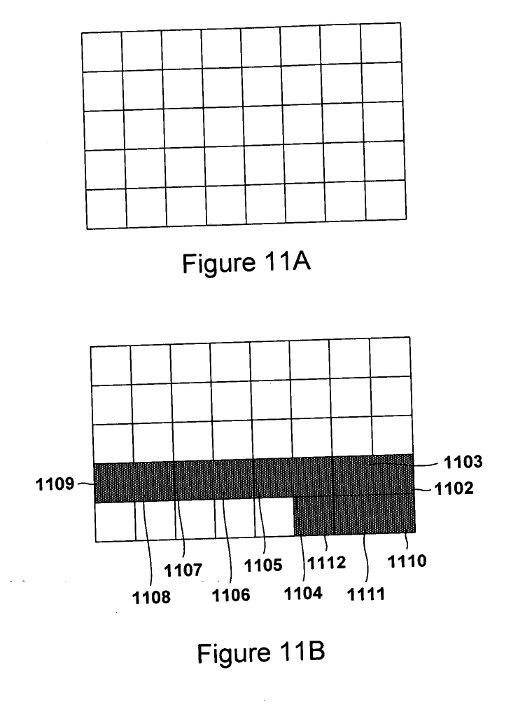

A third type of CGH is referred to as microarray-based CGH (“aCGH”). FIGS. 10-11 illustrate microarray-based CGH. In FIG. 10, synthetic probe oligonucleotides having sequences equal to contiguous subsequences of hypothetical chromosome 407 and/or 408 in the hypothetical, normal genome illustrated in FIG. 4, are prepared as features on the surface of the microarray 1002. For example, a synthetic probe oligonucleotide having the sequence of one strand of the region 1004 of chromosome 407 and/or 408 is synthesized in feature 1006 of the hypothetical microarray 1002. Similarly, an oligonucleotide probe corresponding to subsequence 1008 of chromosome 407 and/408 is synthesized to produce the oligonucleotide probe molecules of feature 1010 of microarray 1002. In actual cases, probe molecules may be much shorter relative to the length of the chromosome, and multiple, different, overlapping and non-overlapping probes/features may target a particular gene. Nonetheless, there is a definite, well-known correspondence between microarray features and genes.

The microarray may be exposed to sample solutions containing fragments of DNA. In one version of aCGH, an array may be exposed to fragments, labeled with a first chromophore, prepared from abnormal tissue and to fragments, labeled with a second chromophore, prepared from normal tissue. The normalized ratio of signal emitted from the first chromophore versus signal emitted from the second chromophore for each feature provides a measure of the relative abundance of the portion of the normal chromosome corresponding to the feature in the abnormal tissue versus the normal tissue. In the hypothetical microarray 1002 of FIG. 10, each feature corresponds to a different interval along the length of chromosome 407 and/408 in the hypothetical wild-type genome illustrated in FIG. 4. When fragments prepared from a normal tissue sample, labeled with a first chromophore, and DNA fragments prepared from normal tissue labeled with the second chromophore, are both hybridized to the hypothetical microarray shown in FIG. 10, and normalized intensity ratios for light emitted by the first and second chromophores are determined, the normalized ratios for all features should be relatively uniformly equal to one.

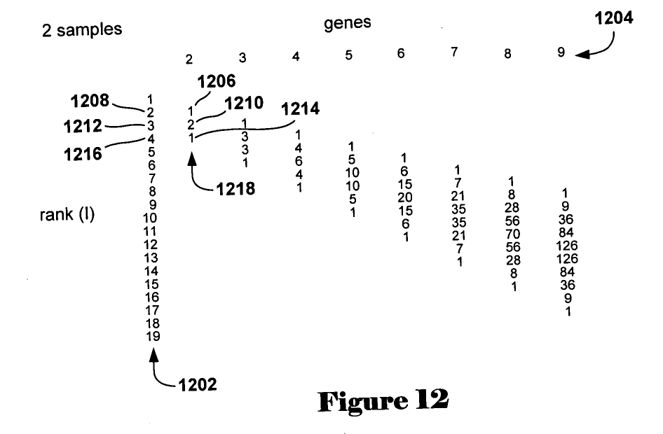

FIG. 11 represents an aCGH data set for two normal, differentially labeled samples hybridized to the hypothetical microarray shown in FIG. 10. The normalized ratios of signal intensities from the first and second chromophores are all approximately unity, shown in FIG. 11, by log ratios for all features of the hypothetical microarray 1002 displayed in the same color. By contrast, when DNA fragments isolated from tissues having the mutant genotype, illustrated in FIG. 5, labeled with a first chromophore are hybridized to the microarray, and DNA fragments prepared from normal tissue, labeled with a second chromophore, are hybridized to the microarray, then the ratios of signal intensities of the first chromophore versus the second chromophore vary significantly from unity in those features containing probe molecules equal to, or complementary to, subsequences of the amplified genes 430, 431, and 432. As shown in FIG. 12, increase in the ratio of signal intensities from the first and second chromophores, indicated by darkened features, are observed in those features 1202-1212 with probe molecules equal to, or complementary to, subsequences spanning the amplified genes 430, 431, and 432. Similarly, a decrease in signal intensity ratios indicates gene deletion in the abnormal tissues.

Microarray-based CGH data obtained from well-designed microarray experiments provide a relatively precise measure of the relative or absolute number of copies of genes in cells of a sample tissue. Sets of aCGH data obtained from pre-cancerous and cancerous tissues at different points in time can be used to monitor genome instability in particular pre-cancerous and cancerous tissues. Quantified genome instability can then be used to detect and follow the course of particular types of cancers. Moreover, quantified genome instabilities in different types of cancerous tissue can be compared in order to elucidate common chromosomal abnormalities, including gene amplifications and gene deletions, characteristic of different classes of cancers and pre-cancerous conditions. Unfortunately, biological data can be extremely noisy, with the noise obscuring underlying trends and patterns. Scientists, diagnosticians, and other professionals have therefore recognized a need for statistical methods for normalizing and analyzing aCGH data, in particular, and CGH data in general, in order to identify signals and patterns indicative of chromosomal abnormalities that may be obscured by noise arising from many different kinds of experimental and instrumental variations.

One approach to ameliorating the effects of high noise levels in CGH data involves, as a first step, normalizing sample-signal data by using control signal data. In many aCGH experiments, normal, control samples, including chromosomal DNA fragments of chromosomal DNA fragments, isolated from normal tissues are hybridized to arrays as control samples along with DNA fragments or copies isolated or produced from abnormal or diseased tissues for which a measure of chromosomal alterations or abnormalities is sought. Often, multiple control samples are available. Therefore, rather than simply using the log ratio of the signal generated by hybridization of fragments from diseased tissue to signal generated from one control sample, the signal generated from diseased tissue can be normalized using multiple control-sample-derived signals. It should be noted that the methods of the present invention may be applied to normalization of any signals produced from any type of sample, including diseased-tissue samples, samples produced by particular experiments, samples produced at particular times during particular experiments, and other samples of interest. The phrase “diseased tissue sample” is therefore interchangeable, in the following discussions, with the phrase “sample of interest.”

In a more general case, an aCGH array may contain a number of different features, each feature generally containing a particular type of probe, each probe targeting a particular chromosomal DNA subsequence indexed by index k that representis a genomic location. A subsequence indexed by index k is referred to as “subsequence k.” One can define the signal generated for subsequence k by either a control or diseased-tissue sample j as the sum of the log-ratio signals from the different probes targeting subsequence k divided by the number of probes targeting subsequence k or, in other words, the average log-ratio signal value generated from the probes targeting subsequence k, as follows:

$$C ( k , j ) = \frac{{\sum }_{b ∈ \{ features containing probes for k \}}^{} C ( b , j )}{{num\_features}_{k}}$$

where num_featuresₖ is the number of features that target the subsequence k; and

C(b,j) is the normalized signal log ratio for sample j at feature b.

In the case where a single probe targets a particular subsequence, k, then no averaging is needed. In the following discussion, normalization of signals for a solution of interest is discussed, such as a solution of DNA fragments obtained from a particular tissue or experiment. A solution of interest may be subject to a single CGH analysis, or a number of identical samples derived from the solution of interest may be each separately subject to CGH analysis, and the signals produced by the analysis for each subsequence k may be averaged to produce a single, averaged, signal data set for the solution of interest.

To re-emphasize, each aCGH data point is generally a log ratio of signals read from a particular feature of a microarray that contains probes targeting a particular subsequence, the log-ratio of signals representing the ratio of signals emitted from a first label used to label fragments of a diseased tissue to a signal generated from a second label used to label fragments of a normal, control tissue. Both the diseased-tissue fragments and the normal, control fragments hybridize to normal-tissue-derived probe molecules on the microarray. A normal tissue or sample may be any tissue or sample selected as a control tissue or sample for a particular experiment. The term “normal” does not necessarily imply that the tissue or sample represents a population average, a non-diseased tissue, or any other subjective or object classification.

Having averaged signals produced from features containing identical probes, and having obtained a single, or a single averaged, data set for a solution of interest, such as for a particular diseased tissue, and having obtained multiple, control data sets, the multiple, control data sets can be used together to normalize the data set for the solution of interest in order to generate better signal-to-noise ratios for subsequence amplification and deletion indications, and indications of other sequence abnormalities. Using multiple control data sets for normalization, rather than a single control data set, produces more statistically reliable indications of sequence abnormalities.

Next, a mean control-signal for a particular subsequence k can be computed from the signal generated for subsequence k by a number J of control samples 1, . . . , J as follows:

$${μ}_{k} = \frac{{\sum }_{j = 1}^{J} C ( k , j )}{J}$$

where J=number of normal, control samples

Similarly, the standard deviation for the J control signals for subsequence k can be computed as follows:

$${σ}_{k} = \sqrt{\frac{{\sum }_{j = 1}^{J} {( {μ}_{k} - C ( k , j ) )}^{2}}{J - 1}}$$

Using μₖ and σₖ, a normalized signal for a particular subsequence k generated by a diseased-tissue sample s can be computed as:

$${C}_{z} ( k , s ) = \frac{C ( k , s ) - {μ}_{k}}{{σ}_{k}}$$

In cases where there are not a sufficient number of control sample signals in order to compute a reliable mean and standard deviation for generation of the normalized signal for a particular diseased-tissue sample C_{z}(k, s), a rank-ordering-based normalization may be carried out. First, the position of an element q within an ordered set of values X, such that q ε X, is defined, as follows:
position(q, X)=i
where X={x₁,x₂, . . . , xₘ};
x₁≦x₂≦x₃ . . . ≦xₘ;
and q=x₁

The normalized signal produced by diseased-tissue-sample s for a particular subsequence k is the position, or rank, of the signal generated for the subsequence k by diseased-tissue sample s within the ordered set C that includes a number of signals generated by control samples j₁, . . . j_{J} as well as by the diseased-tissue sample s, as follows:
Cᵣ(k,s)=position(C(k,s),C)
where s=a particular sample; and
C={C(k,j₁),C(k,j₂), . . . , C(k,j_{J})}∪C(k,s)

Thus, as discussed above, one can compute either a mean-and-standard-deviation-based normalized diseased-tissue signal for a particular subsequence k, C_{z}, or a rank-order-based normalized signal generated from a diseased-tissue sample s, Cᵣ. The former normalization is used when there are sufficient number of control samples to determine a statistically reliable mean and standard deviation. Otherwise, the rank-order method is employed.

Subsequence deletions and amplifications generally span a number of contiguous subsequences of interest, such as genes, control regions, or other identified subsequences, along a chromosome. It therefore makes sense to analyze aCGH data in a chromosome-by-chromosome fashion, statistically considering groups of consecutive subsequences along the length of the chromosome in order to more reliably detect amplification and deletion. Specifically, it is assumed that the noise of measurement is independent for each subsequence along the chromosome, and independent for distinct probes. Statistical measures are employed to identify sets of consecutive subsequences for which deletion or amplification is relatively strongly indicated. This tends to ameliorate the effects of spurious, single-probe anomalies in the data. A parametric approach can be used when the measurement noise along the chromosome is independent for distinct probes and aproximately normally distributed. A non-parametric approach is used when these assumptions cannot be made.

For either method, one considers the measured, normalized, or otherwise processed signals for subsequences along the chromosome of interest to be a vector V as follows:
V={v₁,v₂, . . . ,vₙ}
where vₖ=C_{z}(k,s)or vₖ=Cᵣ(k, s)

Note that the vector, or set V, is sequentially ordered by position of subsequences along the chromosome. In the parametric approach, a statistic S is computed for each interval I of subsequences with fixed size along the chromosome as follows:

$$S ( I ) = ( {\sum }_{k = i , … , j}^{} {v}_{k} ) \cdot \frac{1}{\sqrt{j - i + 1}}$$

where I={v₁, . . . ,vⱼ}; and
vₖ=C_{z}(k,s)

Under a null model assuming no sequence aberrations, the statistic S has a normal distribution of values with mean=0 and variance=1, independent of the number of probes included in each interval I. The statistical significance of the normalized signals for the subsequences in an interval I can be computed by a standard probability calculation based on the area under the normal distribution curve:

$$Prob ( \vert S ( I ) \vert > z ) \approx ( \frac{1}{\sqrt{2 π}} ) \frac{1}{z} {ⅇ}^{\frac{{z}^{2}}{2}}$$

Alternatively, the magnitude of S(I) can be used as a basis for determining alteration.

A non-parametric approach employs the rank-order-based normalized signal values for a diseased-tissue sample and a number of control samples. A rank-sum can be computed for a given interval I by adding together the rank-order-based normalized signals for each of the subsequences v₁, . . . vₖ, and the expected value for the rank of an interval rank (I) is straightforwardly computed, as follows:

$$Exp ( rank ( I ) ) = \frac{m + 1}{2} \cdot d$$

$$where$$

$$I = \{ {v}_{i} , … , {v}_{j} \} ;$$

$${v}_{l} = {C}_{r} ( l , s ) ; \\ m = number of control samples + 1 ;$$

$$rank ( I ) = {\sum }_{l = i to j}^{} {v}_{l} ;$$

$$and$$

$$d = j - i + 1.$$

In order to statistically consider and evaluate intervals for putative amplification and deletion, one needs to compute the probability of large deviations from the expected value. To do this, the k-th order convolution of the uniform distribution on {1, . . . ,m} is computed. The probability Tₘ(r,z) is the probability that r independent random variables uniformly distributed in {1, . . . ,m} sum to exactly the value z. This probability can be recursively computed as follows:

$${T}_{m} ( 1 , z ) = \{ \begin{matrix}\frac{1}{m} & z ∈ \{ 1 , … , m \} \\ 0 & z ∉ \{ 1 , … , m \}\end{matrix} \}$$

$${T}_{m} ( r , z ) = {\sum }_{w = z - m , … , z - 1}^{} \frac{{T}_{m} ( r - 1 , w )}{m}$$

The exact probabilities Tₘ(r,z) can be used to compute the probability that a sum of r independent random variables X₁, . . . , Xᵣ uniformly distributed in {1, . . . ,m} is greater than a particular value y, r≦y≦r·m, as follows:

$$Prob ( {\sum }_{i = 1}^{r} {X}_{i} > y ) = {\sum }_{n = y + 1}^{r \cdot m} {T}_{m} ( r , n )$$

A similar sum of Tₘ(r,z) exact probabilities can be used to compute the probability that a sum of r independent random variables uniformly distributed in {1, . . . ,m} is less than a particular value y, r≦y≦r·m, or within an arbitrary range of values.

In a fashion similar to the probability computation using the parametric approach, discussed above, the probability that a sum of random variables, each uniformly distributed from 1 to m, is greater than an observed rank (I) can be used to compute the statistical significance of a relatively high rank (I) value corresponding to an amplification of subsequences within an interval I, as follows:

$$Prob ( {Z}_{m , u} > rank ( I ) )$$

$$where$$

$$I = \{ {v}_{i} , … , {v}_{j} \} ;$$

$$μ = j - i + 1 ; \\ m = number of control samples + 1 ;$$

$${X}_{i} = an independent , random variable uniformly distributed from 1 to m ;$$

$$and$$

$${Z}_{m , u} = {\sum }_{i = 1 to u}^{} {X}_{i}$$

Similarly, the probability that the sum of the number of random variables uniformly distributed from 1 to m is less than an observed rank (I) can be used to compute the significance of a relatively low rank (I) value indicating deletion of the subsequences in interval (I), as follows:

$$Prob ( {Z}_{m , u} < rank ( I ) )$$

$$where$$

$$I = \{ {v}_{i} , … , {v}_{j} \} ;$$

$$u = j - i + 1 ;$$

$$m = number of control samples + 1 ;$$

$${X}_{i} = an independent , random variable uniformly distrubuted from 1 to m ; and {Z}_{m , u} = {\sum }_{i = 1 to u}$$

It should be noted that various different interval lengths may be used, iteratively, to compute amplification and deletion probabilities over a particular biopolymer sequence. In other words, a range of interval sizes can be used to refine amplification and deletion indications over the biopolymer.

As an example of the computation of the above-described probabilities for determining significance values for computed interval ranks, the following C++-like pseudocode can be used to determine the probability of observing a rank (I) value for some numbers of control samples plus a diseased-tissue sample for an arbitrary number of subsequences in I within a range of rank (I) values. This concise C++-like pseudocode is included in order to illustrate one approach to computing probabilities of ranges of rank (I) values, in turn used to estimate the significance of an observed rank (I) value in an experimental procedure. It is not presented as the most efficient or most elegant approach to the problem.

First, a small number of constants are declared:

1 const int MAX_SAMPLES = 6;
2 const int MAX_GENES = 9;
3 const int TABLE_LENGTH =
4 MAX_GENES * (MAX_SAMPLES / 2) * (MAX_SAMPLES + 1);

These constants specify the maximum number of samples and subsequences that can be specified as initial values with a probability determination.

Next, a declaration for a simple class “createTable” is provided:

```
  1 class createTable
  2 {
  3  private:
  4  int rank;
  5  int nGenes;
  6  int nSamples;
  7  int accumulator;
  8  double probs[TABLE_LENGTH][MAX_GENES − 1];
  9  int sampleSizePtrs[MAX_SAMPLES + 1];
10
11  public:
12  int compute(int Rank, int Genes, int Samples);
13  void recCompute(int r, int sum);
14  void pTable( );
15  double Prob(int numGenes, int numSamples, int startZ, int endZ);
16 };
```

The class “createTable” creates a table of counts of the number of possible rank combinations that lead to a particular rank (I) value for a given number of subsequences in interval I for a particular number of samples m. The private data members for the class “createTable” include: (1) rank, a particular rank (I) value; (2) nGenes, the number of subsequences an interval I; (3) nSamples, a number of samples in the experiment; (4) accumulator, an integer used to accumulate counts in a recursive routine, described below; (5) probs, a table of probabilities obtained by dividing the number of combinations of ranks leading to a particular rank (I) value divided by the total number of possible combinations of subsequence-rank values; and (6) sampleSizePtrs, a table of indexes into the table “probs,” described above. The class “createTable” includes the following function members: (1) compute, a routine that computes the probability of a particular rank (I) for a particular number of subsequences over a particular number of samples; (2) recCompute, a recursive routine called by the routine “compute” for computing the counts of the combinations of subsequence-rank values that sum to a particular rank (I) value; (3) pTable, a routine that computes the probability values stored in the table “probs,” described above; and (4) Prob, a routine that computes the probability that an observed rank (I) value falls within a range of rank (I) values specified as arguments for a particular number of subsequences over a particular number of samples. Next, an implementation of the recursive routine “recCompute” is provided:

```
  1 void createTable::recCompute(int r, int sum)
  2 {
  3  int i, j;
  4  int range;
  5
  6  range = rank − (nGenes−r) − sum;
  7  if (range > nSamples) range = nSamples;
  8
  9  if (r == nGenes − 1)
10  {
11  for (i = 1; i <= range; i++)
12  {
13  j = rank − (sum + i);
14  if (j <= nSamples) accumulator ++;
15  }
16  }
17  else
18  for (i = 1; i <= range; i++) recCompute(r+1, sum + i);
19 }
```

The recursive routine “recCompute” recursively computes the number of combinations of subsequence-rank values that can produce a particular rank (I) value. It recursively considers the possible subsequence-rank values for each subsequence in an interval.

Next, an implementation for the routine “Compute” is provided:

```
  1 int createTable::compute(int Rank, int Genes, int Samples)
  2 {
  3  if ((Rank < Genes) || (Rank > (Genes * Samples))) return 0;
  4  else
  5  {
  6  nGenes = Genes;
  7  rank = Rank;
  8  nSamples = Samples;
  9  accumulator = 0;
10  recCompute(1,0);
11  return accumulator;
12  }
13 }
```

The routine “compute” returns either 0, in the case that the specified rank does not fall within the range of possible ranks for the specified number of subsequences and samples, or otherwise calls recursive routine “recCompute” to compute the number of combinations of subsequence-rank values leading to a particular rank, specified as an argument. Next, an implementation for the routine “pTable” is provided:

```
  1 void createTable::pTable( )
  2 {
  3  int zz, numGenes, numSamples, curPtr = 0;
  4  double count;
  5  double pb;
  6  for (numSamples = 2; numSamples <= MAX_SAMPLES;
    numsamples++)
  7  {
  8  sampleSizePtrs[numSamples] = curPtr;
  9  for (zz = 2; zz <= (MAX_GENES * numSamples); zz++)
10  {
11  for (numGenes = 2; numGenes <= MAX_GENES;
        numGenes++)
12  {
13  count = compute(zz, numGenes, numSamples);
14  pb = count / pow(numSamples, numGenes);
15  probs[curPtr][numGenes − 2] = pb;
16  }
17  curPtr++;
18  }
19  }
20 }
```

This routine computes the probabilities of observing a particular rank (I) value by dividing the number of combinations for the rank (I) value computed by the routine “Compute,” on line 13 by the total number of combinations of subsequence-rank values, computed on line 14.

Next, an implementation of the routine “Prob” is provided:

```
  1 double createTable::Prob(int numGenes, int numSamples, int startZ,
int endZ)
  2 {
  3  double acc = 0;
  4  int max = numSamples * numGenes;
  5  int table = sampleSizePtrs[numSamples];
  6
  7  if (startZ < 2) startZ = 2;
  8  if (endZ > max) endZ = max;
  9
10  for (int i = table + startZ − 2; i < table + endZ − 1; i++)
11  acc += probs[i][numGenes − 2];
12  return acc;
13 }
```

This routine simply sums the probabilities of individual rank (I) values within a range of rank (I) values in order to compute the probability of observing a particular rank (I) value within a range of rank (I) values.

Finally, a simple main routine is provided to indicate how a probability is computed using an instance of the class “createTable”:

1 int main(int argc, char* argv[])
2 {
3 createTable c;
4 double res;
5
6 c.pTable( );
7 res = c.Prob(8,5,8,40);
8 return 0;
9 }

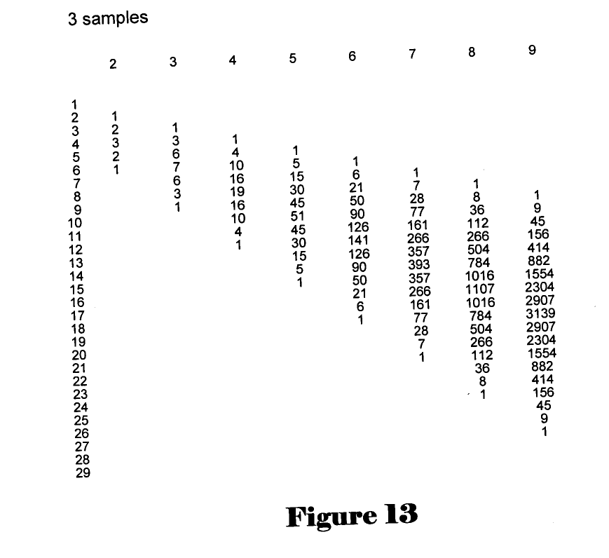

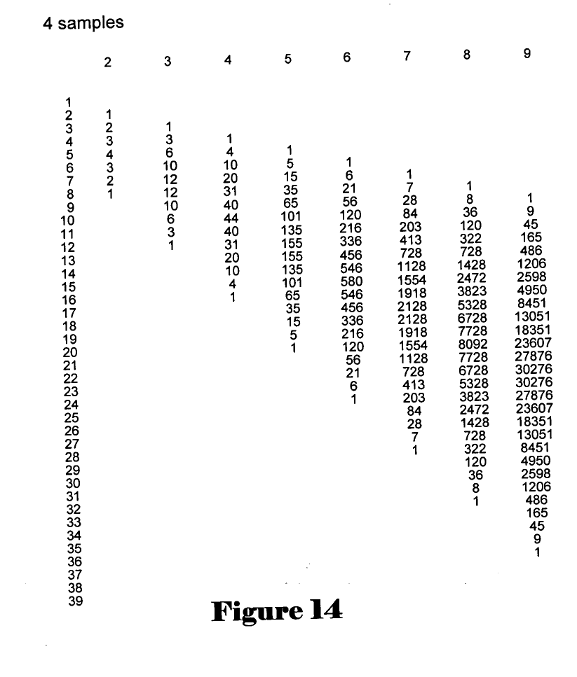

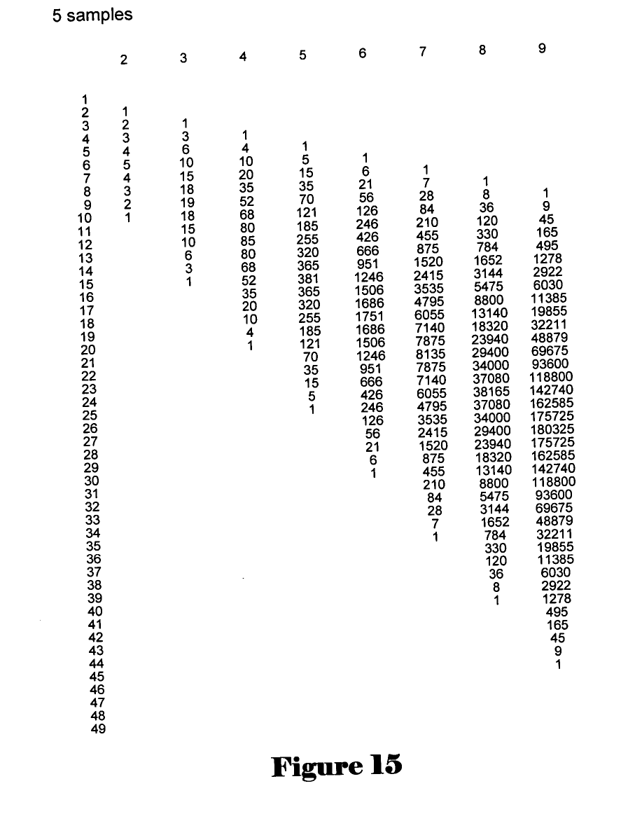

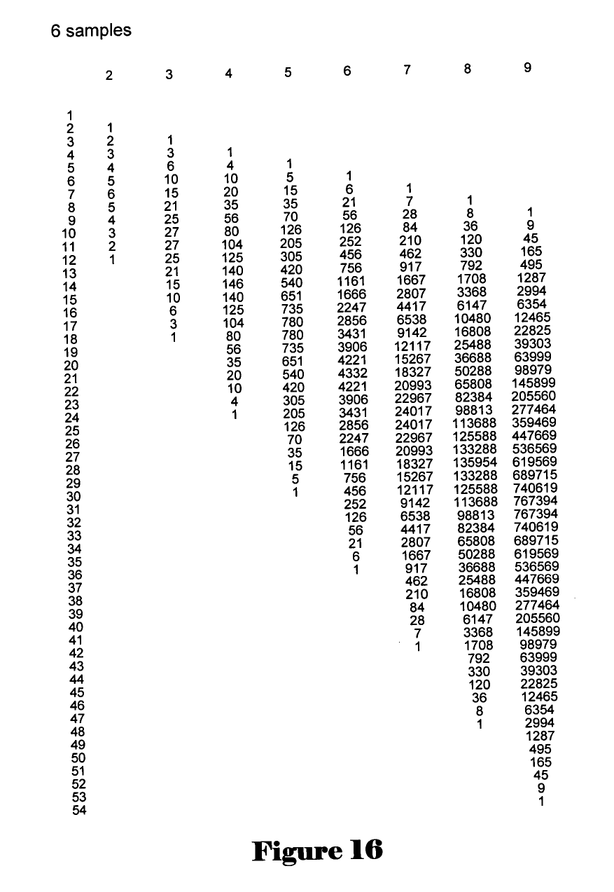

FIGS. 12-16 show data generated from a program like the above C++-like pseudocode that illustrates the number of combinations of subsequence-rank values that lead to a particular rank (I) value for a number of subsequences in an interval and an arbitrary number of samples. All five figures use the same illustration conventions, described only for FIG. 12, in the interest of brevity. In FIG. 12, the combinations for various arbitrary numbers of subsequences and samples are shown. FIGS. 13-16 show the combinations for three through six samples. Column 1202 lists possible rank (I) values, and horizontal axis 1204 is incremented in the number of subsequences in a particular interval, from two to nine subsequences. Zero values are shown as blanks in FIG. 12-15. For example, for an interval of two subsequences, there is one 1206 combination of subsequence-rank values that lead to a rank (I) value of 2 1208, two combinations 1210 of subsequence-rank values that lead to a rank (I) value of 3 1212, and one combination 1214 that leads to a rank (I) value 1216 of 4. The total number of combinations for a particular number of subsequences in samples can be obtained by adding all combinations in a particular column in the figure. The same value can be computed as the number of samples raised to a power equal to the number of subsequences. Thus, for the first column 1218 of data in FIG. 12, the total number of combinations is 1+2+1=2²=4. The probabilities computed by the above pseudocode implementation can be attained by summing the combinations within a column corresponding to the ranks within a desired range and dividing by the total number of combinations represented by the column.

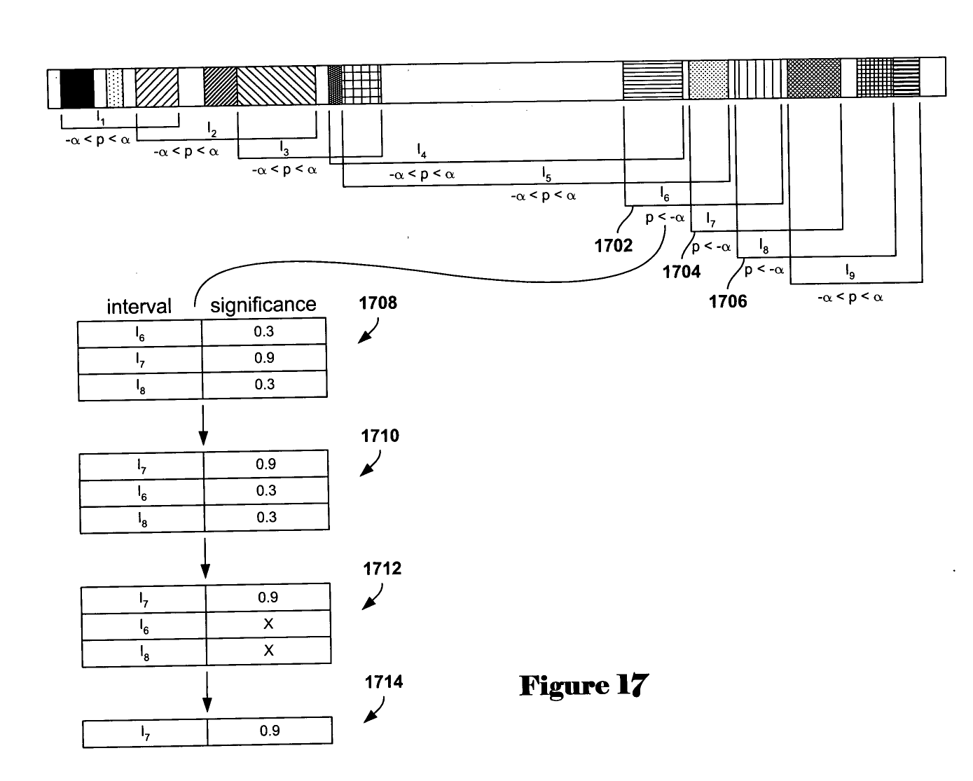

After the probabilities for observing either the parametric, statistical value for intervals or the rank values for intervals are computed, those intervals with computed probabilities outside of a reasonable range of expected probabilities under the null hypothesis of no amplification or deletion are identified, and redundancies in the list of identified intervals are removed. FIG. 17 illustrates one method for identifying and ranking intervals and removing redundancies from lists of intervals identified as corresponding to probable deletions or amplifications. In FIG. 17, the intervals for which probabilities are computed along the chromosome C₁(402 in FIG. 4) for diseased tissue with an abnormal chromosome (502 in FIG. 5) are shown. Each interval is labeled by an interval number, Iₓ, where x ranges from 1 to 9. For most intervals, the calculated probability falls within a range of probabilities consonant with the null hypothesis. In other words, neither amplification nor deletion is indicated for most of the intervals. However, for intervals I₆ 1702, I₇, 1704, and I₈, 1706, the computed probabilities fall below the range of probabilities expected for the null hypothesis, indicating potential subsequence deletion in the diseased-tissue sample. These three intervals are placed into an initial list 1708 which is ordered by the significance of the computed probability into an ordered list 1710. Note that interval I₇ 1704 exactly includes those subsequences deleted in the diseased-tissue chromosome (502 in FIG. 5), and therefore reasonably has the highest significance with respect to falling outside the probability range of the null hypothesis. Next, all intervals overlapping an interval occurring higher in the ordered list are removed, as shown in list 1712, where overlapping intervals I₆ and I₈, with less significance, are removed, as indicated by the character X placed into the significance column for the entries corresponding to intervals I₆ and I₈. The end result is a list containing a single interval 1714 that indicates the interval most likely coinciding with the deletion. The final list for real chromosomes, containing thousands of subsequences and analyzed using hundreds of intervals, may generally contain more than a single entry.

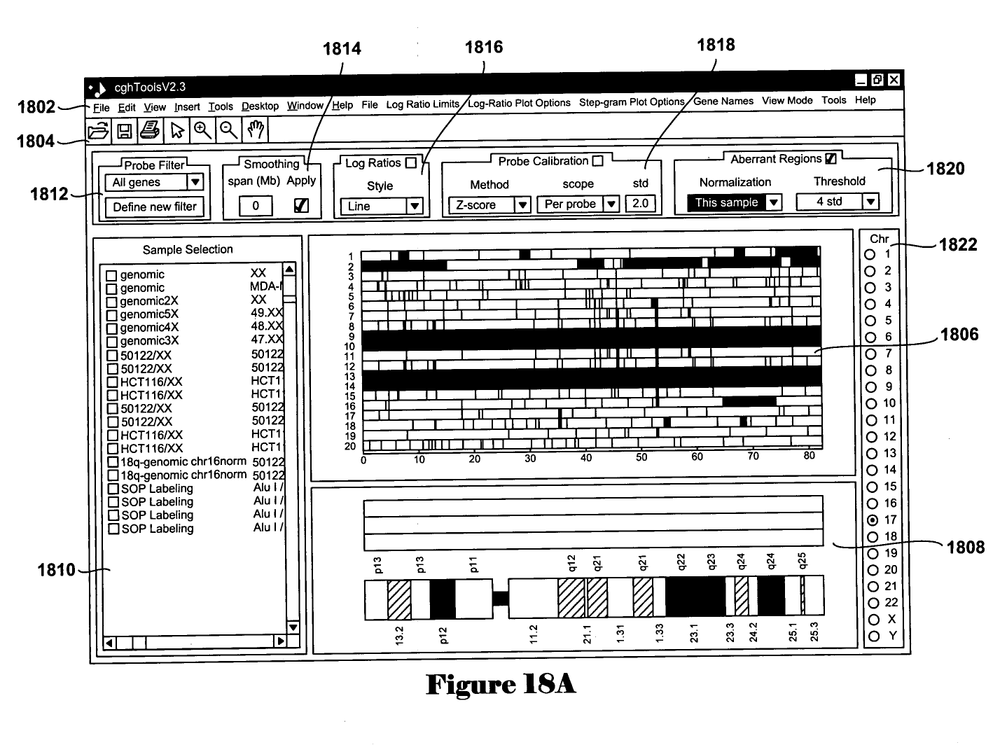

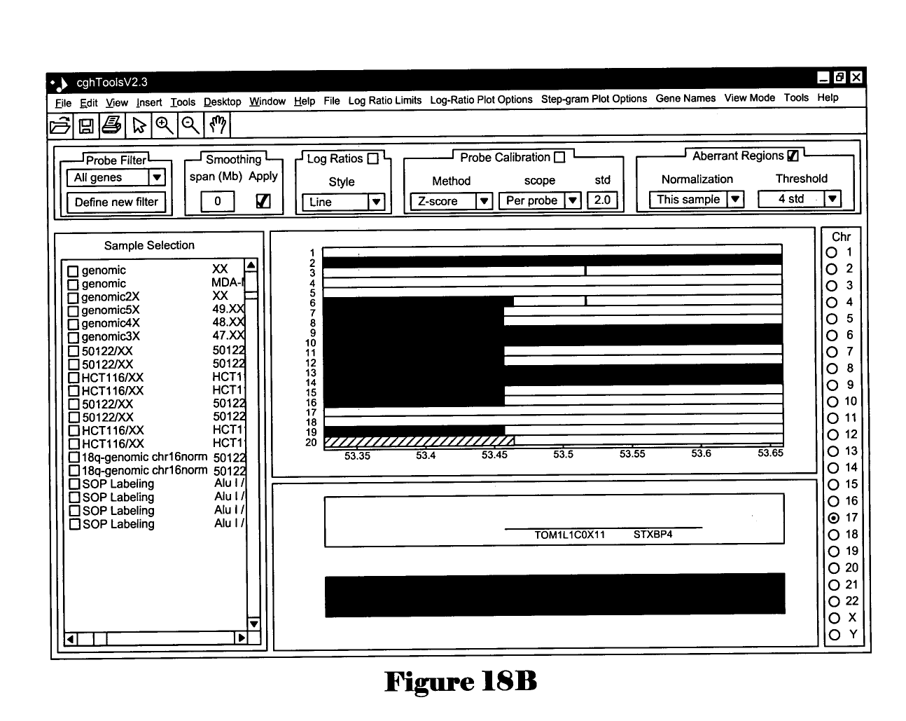

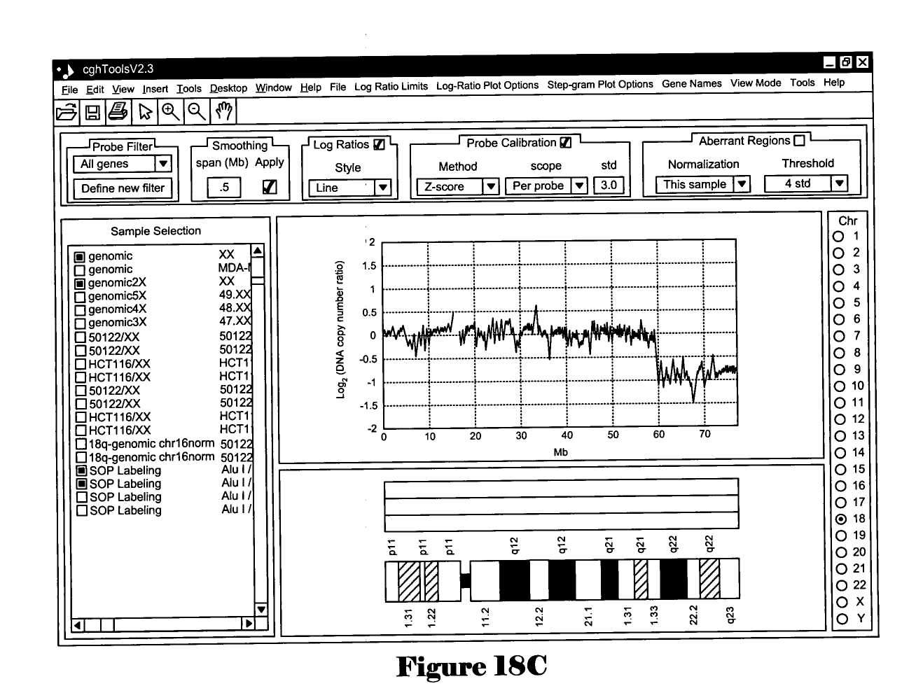

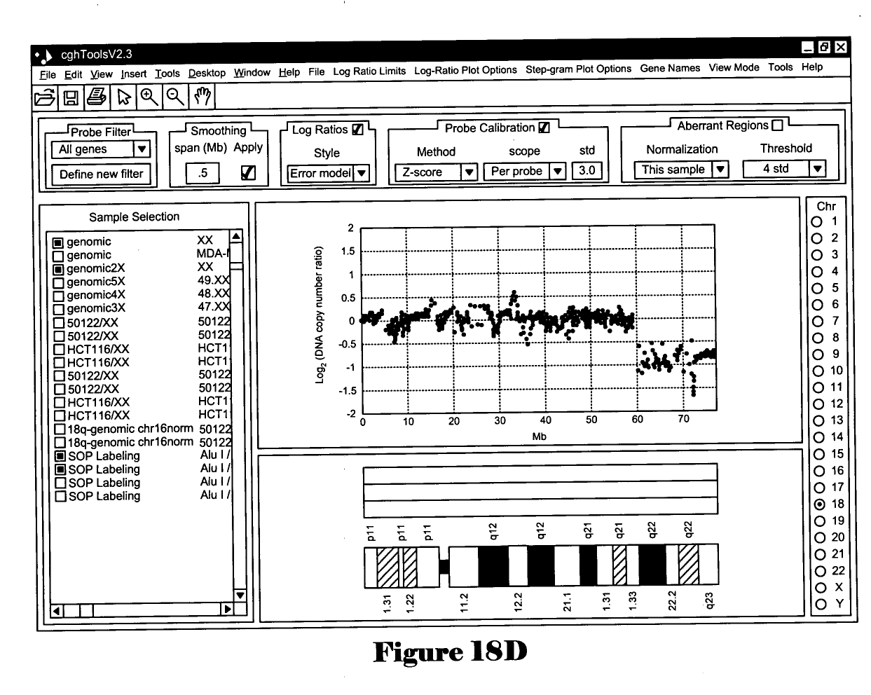

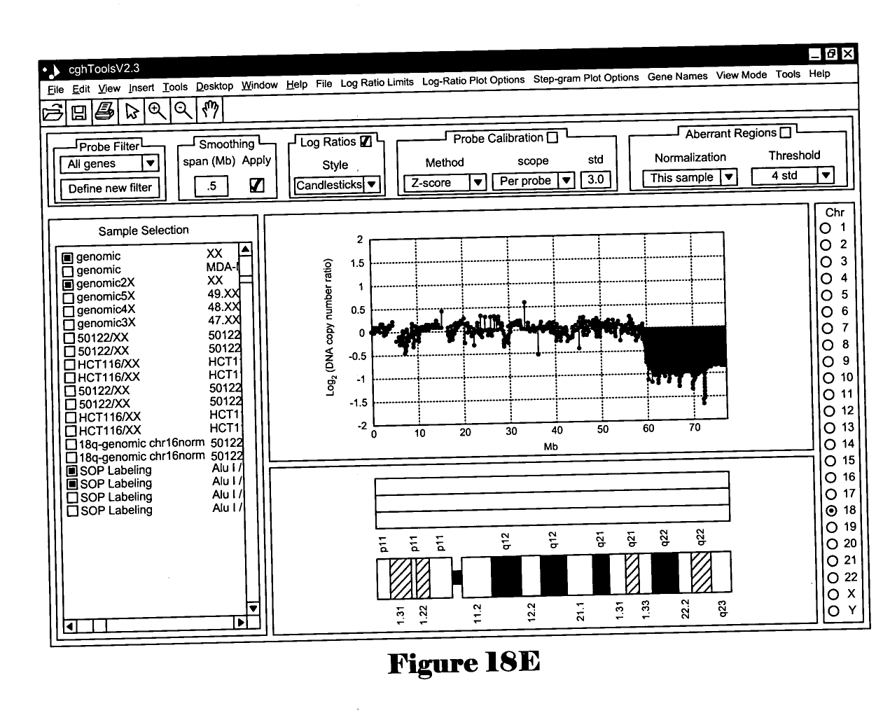

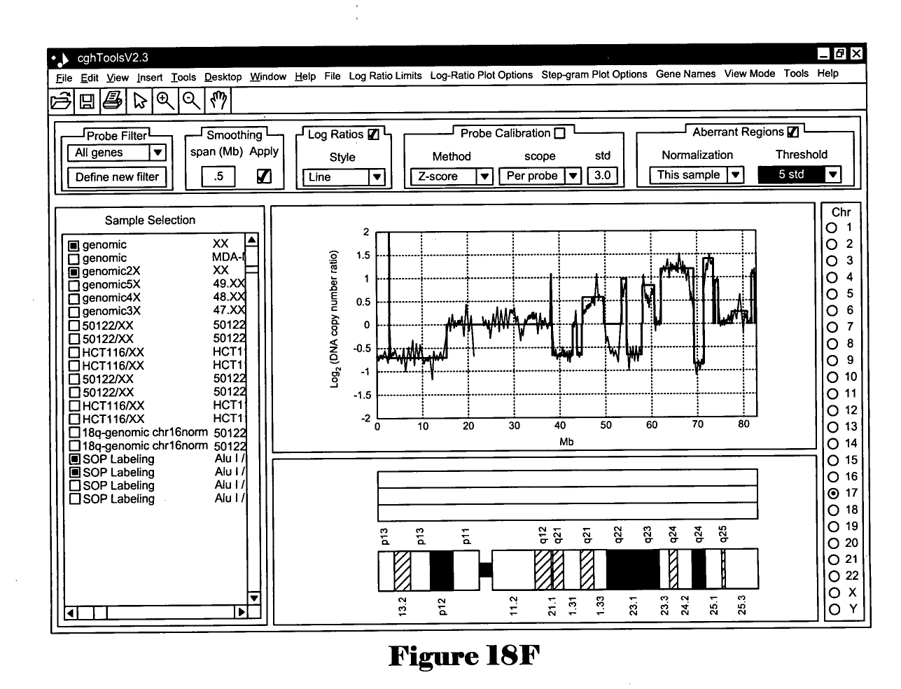

FIGS. 18A-F show screen captures that illustrate a user interface developed to provide visual and interactive access to methods of CGH data analysis and results of the analysis as part of a CGH-data-analysis system. Features of the user interface, as shown in FIG. 18A, are first described. FIGS. 8B-F show different displays of the data as controlled through features of the user interface. Features of he user interface include: (1) menu bars 1802-1804, which provide standard operating-system interfaces, data-processing and display options, user-assistance interfaces, and other standard functionalities; (2) a data-analysis-representation display area, in which analysis of CGH data is displayed in different ways, including heat-map representations; (3) an annotation window 1808 that, concurrently with display of CGH-data analysis, in data-analysis-representation display area 1806, provides textual and graphical annotation of the biopolymer subsequences, analysis of which are displayed in data-analysis-representation display area 1806, annotations including gene names, gene product names, and other genomic information related to a genomic regions including the biopolymer sequences; (4) a sample-selection window 1810, that displays, and provides for user selection of, various samples to be analyzed; (5) a probe-filter-selection window 1812, that allows for selection of all or a subset of the probes used to generate a CGH data set; (6) a smoothing-selection window 1814 that allows for selecting the size of subsequence intervals I over which to compute statistics; (7) a log-ratio-representation-selection window 1816 that controls the style of display of log ratios in the data-analysis-representation display area 1806; (8) a probe-calibration-selection window 1818, that allows for application of parametric or non-parametric statistics, and selection of various parameters that control the exact analysis method, from among the above-described analysis methods, and other methods, for analyzing CGH data; (9) an aberrant-regions-selection window 1820 that provides further parameters for controlling the exact analytical method applied to the CGH data; (10) a genomic-range selection bar that allows a user to select a range of genomic locations for display using a mouse click for each end of the range to zoom the display into the range, as well as allowing a user to select a broader range than the currently displayed range; and (11) a chromosome-selection column that allows individual chromosomes to be selected for analysis.

The data-analysis-representation display area 1806 displays, along selected regions of a chromosome or entire genome, in the case of DNA biopolymer analysis, a heat-map representation of the results of a CGH data analysis for each of a number of samples, indicating with increasing intensity of one color, such as green, the likelihood that a region is deleted, and indicating with increasing intensity of a different color, such as red, the likelihood that a region is amplified. In the heat-map representation, regions in which neither amplification or deletion are indicated may be represented in a neutral color, such as white or grey. The CGH analysis is undertaken, as described above, to use control data, and to compute deletion and amplification statistics that factor in indications of adjoining subsequences and the various diseased tissue samples selected in the sample-selection window 1810. As FIG. 18B indicates, the range of display may be decreased to zoom in on a particular region of a genome or chromosome.

FIGS. 18C-F show different display formats for single sample signals, and sample signals in the context of control data. In FIG. 18C, for example, a displayed line represents the computed signal log ratio for a sample of interest within a background, or control patch, representing a range of control signal data about the mean control signal data. Thus, as shown in Figure C, a deletion is easily recognized by the displayed line falling below the control patch. To enhance visibility of deletions and amplifications, the portions of a line representation of sample-of-interest signal data that falls below or above the control patch can be differentially colored, for example green and red, respectively, when the line representing sample-of-interest data within the control patch is colored black.

Although the present invention has been described in terms of a particular embodiment, it is not intended that the invention be limited to this embodiment. Modifications within the spirit of the invention will be apparent to those skilled in the art. For example, an almost limitless number of different implementations of computer programs and computer-program routines can be created to compute the above-described analysis methods for analyzing chromosomal aberrations in diseased-tissue samples when a number of control samples are available. Although recursive methods are indicated in the above discussion, and used in the above C++-like pseudocode implementation, more efficient, non-recursive algorithms can be employed to more efficiently compute the desired statistics. The above-described methods can be easily modified to encompass experimental data from many different organisms having different numbers of chromosomes, different numbers of subsequences per chromosome, and other genetic differences. In each component of the above-described method, many possible mathematically similar, but alternative approaches may be employed. For example, different methods for computing means and variances can be used, as well as different statistical parameters used to characterize particular distributions. Many different types of user-interface implementations, in addition to the user-interface implementation discussed above with reference to FIGS. 18A-F can be employed to allow for convenient selection of parameters that control CGH analysis and various different CGH-data-analysis-results display formats.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purpose of illustration and description. They are not intended to be exhaustive or to limit the invention to the precise forms disclosed. Obviously many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method for normalizing comparative hybridization data collected for a biopolymer sequence, the method comprising:
for a number of subsequences of the biopolymer sequence,
determining a hybridization level for biopolymer fragments, in a particular sample, with a currently considered subsequence;
determining hybridization levels for biopolymer fragments of control samples j₁, . . . ,jₙ with the currently considered subsequence; and
computing a normalized hybridization level for fragments of the particular sample with the currently considered subsequence by determining a difference between the determined hybridization level for biopolymer fragments in the particular sample and a mean computed for the determined hybridization levels for biopolymer fragments of control samples j₁, . . . , jₙ relative to a variance computed for the determined hybridization levels for fragments of control samples j₁, . . . ,jₙ.

2. The method of claim 1 wherein the biopolymer is DNA.

3. The method of claim 2 wherein the comparative hybridization data is obtained from an assay that combines an enrichment phase and a micro-array-based detection phase.

4. The method of claim 2 wherein the data is collected from array-based, comparative-genomic-hybridization experiments.

5. Computer instructions that implement the method of claim 1 stored in a computer readable medium.

6. A comparative hybridization data analysis system that includes hardware-implemented, firmware-implemented, software-implemented, or a combination of two or more of hardware-implemented, firmware-implemented, and software-implemented logic that implements the method of claim 1.

7. A method for normalizing comparative hybridization data collected for a biopolymer sequence, the method comprising:
for a number of subsequences of the biopolymer sequence,
determining a hybridization level for biopolymer fragments, in a particular sample, with a currently considered subsequence;
determining hybridization levels for biopolymer fragments of control samples j₁, . . . ,jₙ with the currently considered subsequence; and
computing a normalized hybridization level for fragments of the particular sample with the currently considered subsequence by
ordering the determined hybridization level for biopolymer fragments in the particular sample and the determined hybridization levels for fragments of control samples j₁, . . . , jₙ to produce an ordered set of determined hybridization-level values, and
selecting a position of the determined hybridization level for biopolymer fragments in the particular sample within the ordered set of values as the normalized hybridization level for biopolymer fragments of the particular sample with the currently considered subsequence.

8. The method of claim 7 wherein the biopolymer is DNA.

9. The method of claim 8 wherein the comparative hybridization data is obtained from an assay that combines an enrichment phase and a micro-array-based detection phase.

10. The method of claim 8 wherein the data is collected from array-based, comparative-genomic-hybridization experiments.

11. Computer instructions that implement the method of claim 7 stored in a computer readable medium.

12. A comparative hybridization data analysis system that includes hardware-implemented, firmware-implemented, software-implemented, or a combination of two or more of hardware-implemented, firmware-implemented, and software-implemented logic that implements the method of claim 7.

13. A method for identifying amplified and deleted regions of a biopolymer sequence obtained from a particular sample, the method comprising:
determining normalized hybridization levels for fragments of the biopolymer sequence, using hybridization levels for fragments of biopolymer sequences obtained from one or more control samples, with respect to each of a set of consecutive subsequences of a standard biopolymer sequence;
storing the determined, normalized hybridization levels as signals in a vector of signals;
generating a set of intervals within the vector of signals;
scoring each interval with a statistical score; and
determining intervals with statistical scores below a first threshold as likely deleted and intervals with statistical scores above a second threshold as likely amplified.

14. The method of claim 13 wherein scoring each interval with a statistical score further includes:
summing signals within each interval and dividing the sum of signals by the square root of the number of signals in the interval to produce a normal statistic S for each interval.

15. The method of claim 14 wherein determining intervals with statistical scores below a first threshold as likely deleted and intervals with statistical scores above a second threshold as likely amplified further includes:
comparing a probability of observing the computed normal statistic for each interval with the first and second thresholds.

16. The method of claim 13 wherein scoring each interval with a statistical score further includes:
summing rank-order-based signals within each interval to produce a rank sum.

17. The method of claim 16 wherein determining intervals with statistical scores below a first threshold as likely deleted and intervals with statistical scores above a second threshold as likely amplified further includes:
comparing a probability of observing the computed rank sum for each interval with the first and second thresholds.

18. The method of claim 13 wherein the biopolymer sequence is a DNA sequence.

19. The method of claim 13 wherein hybridization levels for fragments of the biopolymer sequence are determined by an array-based, comparative hybridization method.

20. Computer instructions that implement the method of claim 13 stored in a computer readable medium.

21. A comparative hybridization data analysis system that includes hardware-implemented, firmware-implemented, software-implemented, or a combination of two or more of hardware-implemented, firmware-implemented, and software-implemented logic that implements the method of claim 13.

22. A user interface provided by a comparative-hybridization data-analysis system comprising:
user-interface features that allow a user to set various parameters to control comparative-hybridization data analysis: and
a data-analysis-representation display area that displays, along selectable regions of a biopolymer sequence, a heat-map representation of the results of a comparative-hybridization data analysis for a selectable number of samples of interest, with graphically encoded indications of amplification, deletion, and other abnormalities.

23. The user interface of claim 22 wherein user-interface features that allow a user to set various parameters to control comparative-hybridization data analysis further include:
a feature that allows a user to select a range of the biopolymer sequence along which to display comparative-hybridization-analysis results;
a feature that allows a user to select one of parametric or non-parametric data normalization;
a feature that allows a user to select one of parametric or non-parametric consecutive-subsequence-based determinations of amplification and deletion probabilities;
a feature that allows a user to select particular samples of interest for analysis; and
a feature that allows a user select one of a number of results-display formats.

24. The user interface of claim 23 wherein results-display formats include a display format in which comparative-hybridization results for a particular sample of interest are displayed overlying a control patch that indicates a corresponding range of values for control results about a mean for the control results.

25. The user interface of claim 23 further including displaying comparative-hybridization results for a particular sample of interest in a first color when the comparative-hybridization results fall within a corresponding range of values for control results, in a second color when the comparative-hybridization results fall above a corresponding range of values for control results, and in a third color when the comparative-hybridization results fall below a corresponding range of values for control results.

26. Computer instructions encoded in a computer readable medium that implement the user interface of claim 22.

27. A comparative hybridization data analysis system that includes hardware-implemented, firmware-implemented, software-implemented, or a combination of two or more of hardware-implemented, firmware-implemented, and software-implemented logic that implements the user interface of claim 22.

28. The user interface of claim 22 wherein selectable regions of a biopolymer sequence include any sequence that can defined by positions of two monomers within the biopolymer sequence.
